(19) United States
(12) Patent Application Publication
(10) Pub. No.: US 2004/0091516 A1
(43) Pub. Date: May 13, 2004

(54) Printing or dispensing a suspension such as three-dimensional printing of dosage forms

(75) Inventors: Charles William Rowe, Medford, MA (US); Wendy E. Pryce Lewis, Watertown, MA (US); Michael J. Cima, Winchester, MA (US); Esteban R.N. Bornancini, North Wales, PA (US); Jill K. Sherwood, Edison, NJ (US); Chen-Chao Wang, West Windsor, NJ (US); Christopher M. Gaylo, Princeton Junction, NJ (US); James A. Fairweather, West Haven, CT (US)

Correspondence Address: SEED INTELLECTUAL PROPERTY LAW GROUP PLLC, 701 FIFTH AVE, SUITE 6300, SEATTLE, WA, 98104-7092, US

(73) Assignees: Therics, Inc., Princeton, NJ; Massachusetts Institute of Technology, Cambridge, MA

(21) Appl. No.: 10/700,783

(22) Filed: Nov. 3, 2003

Related U.S. Application Data
(62) Division of application No. 09/991,556, filed on Nov. 21, 2001, now abandoned.
(60) Provisional application No. 60/339,921, filed on Oct. 29, 2001.
(60) Provisional application No. 60/340,664, filed on Oct. 29, 2001.

(51) Int. Cl. A61L 2/00; B05D 3/00

(52) U.S. Cl. 424/423; 427/2.24

(57) ABSTRACT

The invention includes dispensing a suspension containing solid particles for use in manufacturing a dosage form or other biomedical article by 3DP. The suspension contains solid particles suspended in a liquid. The solid particles may be one or more Active Pharmaceutical Ingredients. The solid particles may be particles of material that are insoluble in the liquid, or they may be particles of a substance that have already dissolved in the liquid up to the saturation level and are present in a concentration beyond what can be dissolved. In addition to solid particles, the liquid may also contain other substances dissolved in it, either substances containing Active Pharmaceutical Ingredients (API) or substances without API. One aspect of the invention includes prevention of agglomeration by adding one or more of several categories of additives to the suspending liquid. Another aspect of the invention includes manipulating the surface charge of the particles in an API suspension to prevent particles from agglomerating. A further aspect of the invention includes an amorphous API that has a greater bioavailability than the corresponding crystalline material. Yet another aspect of the present invention includes a system for providing continuous circulation of the suspension such that the solid particles remain dispersed in the suspension.

### CROSS-REFERENCE TO RELATED APPLICATIONS

This application is a divisional of pending U.S. application Ser. No. 09/991,556, filed Nov. 21, 2001.

This application claims the benefit of Provisional Application filed Oct. 29, 2001, titled “Suspension Printing of Active Pharmaceutical Ingredient,” application number not yet assigned, and Provisional Application filed Oct. 29, 2001, titled “Controlled Release Formulations Containing Three-Dimensional Gradients by Three-Dimensional Printing,” application number not yet assigned; each of which is incorporated herein by reference in its entirety.

### BACKGROUND OF THE INVENTION

1. Field of the Invention

This invention relates to biomedical articles such as oral dosage forms and various forms of implantable biomedical articles, and more particularly, to oral dosage forms manufactured by suspension printing with an active pharmaceutical ingredient.

2. Description of the Related Art

Oral Dosage Forms (ODF) have been most commonly manufactured by a powder pressing operation. Powder pressing is economical and well suited to the production of dosage forms that are of essentially uniform composition.

Some dosage forms require more geometric detail such as nonuniform distribution of substances. Three-dimensional printing allows for controlled placement of substances within the dosage form. Three-dimensional printing is generally described in U.S. Pat. No. 5,204,055, and illustrated in FIG. 1. Dosage forms made by 3DP having complex release

As shown in FIG. 1, drops of a binder liquid 140, 142 are dispensed by a printhead 130, 132 onto a layer of powder 150 by a technique similar to ink-jet printing. Powder particles are joined together by the binder liquid. Subsequent powder layers are sequentially deposited and binder drops dispensed until the desired three-dimensional object is created. Unbound powder supports printed regions until the article is sufficiently dry and then the unbound powder is removed.

When making a dosage form by 3DP, the API has typically been contained in the binder liquid that is dispensed onto the pharmaceutical excipient powder. APIs that are insoluble or only slightly soluble are either not suitable or are extremely difficult to deposit in large amounts via binder liquid into a dosage form made by 3DP. Usually the API is delivered by being dissolved in the binder liquid that is dispensed onto the powder, and the powder is a pharmaceutical excipient containing no API. When the volatile part of the binder liquid evaporates, the previously dissolved API is left behind. The practical limitation of how much API could be delivered into the dosage form was the given API solubility limits.

In 3DP the powder has typically been spread to an overall packing density that approximated 50% solid and 50% void. This packing density leaves only 50% of the total volume of the dosage form that could possibly be filled with binder liquid containing dissolved API. If the binder liquid exactly fills the void space and if for sake of example the API is soluble in the binder liquid to the extent of 20% on a volume basis, which is a fairly high solubility among substances of practical interest, then by filling the empty space completely with binder liquid and allowing the volatile part of the binder liquid to evaporate, 20% of the empty space that could be filled with the API had been dissolved in the binder liquid, therefore, 10% of the overall volume of the powder bed would be API, assuming this very generous solubility. It is possible to re-print the same region with some further benefit, but there is still a significant limitation arising from the solubility limit or maximum concentration of dissolved API that can be contained in the binder liquid.

Many API of interest are only slightly soluble in water or other typical solvents, and so even with multiple printing passes it is difficult to deposit API quantities of practical interest. Traditional solution printing is limited by how much solute can be dissolved in the solvent, as already described. This limit is imposed to avoid having to handle solid particles that have failed to dissolve. Solid particles can settle out resulting in clogging of dispensers and failure to know how much of the substance is actually dispensed. A further limitation is that typically it is not possible to print with a solution which is fully saturated because some unavoidable evaporation of solvent will occur at the nozzle resulting in crystallization of solid at the nozzle tip, which interferes with printing, and so it is necessary to print with a solution whose concentration of solute is somewhat less than saturation.

One alternative to solution printing is suspension printing. Suspensions have sometimes been dispensed through printheads for non-pharmaceutical purposes. For example, some inks (referred to as dye type inks) are solutions, while other inks (referred to as pigment type inks) are suspensions that are typically dilute, such as 5% solids content or less. Such inks have been dispensed through printheads including Continuous-Jet-with Deflection printheads, although such pigment inks do present greater danger than do dye inks of forming clogs and related difficulties. A suspension containing alumina at a volume concentration of 20% was dispensed through a continuous-jet-with-deflection printhead in U.S. Pat. No. 5,387,380. However, these have not involved API.

The problems caused by suspensions in valves that operate by a sealing action of a moving part against a seat is that particles can lodge in places near the seat or can damage components involved in making the seal. Colloidal silica has been dispensed by Bredt in U.S. Pat. No. 5,851,465. However, colloids involve particles that are substantially smaller than those of suspensions and behave differently.

Suspensions of fairly high solids content have been discharged on a continuous basis from orifices for purposes such as depositing layers of powder by slurry deposition for use in 3DP. However, such a simple continuous discharge does not accomplish drop-by-drop selection or drop-on-demand production needed for 3DP.

Further, with respect to biomedical devices other than ODFs, analogous problems exist with respect to dispensing adequate concentrations of a given component. For example, one problem particularly associated with bone substitute manufacturing has been the low strength of the product due to the size of the hydroxyapatite particles spread to form a powder layer in 3DP. Larger particle size has resulted in poor sintering and lower strengths.

### BRIEF DESCRIPTION OF THE SEVERAL VIEWS OF THE DRAWINGS

FIG. 1 is a schematic illustration of three-dimensional printing in accordance with the prior art.

FIG. 2 is a schematic illustration of suspension dispensing through a continuous-jet-with-deflection printhead in accordance with principles of the present invention.

FIG. 3 is an enlarged view of the deflection path within the deflection cell of FIG. 2.

FIG. 4 illustrates multiple printheads of FIG. 2 in parallel in accordance with principles of the present invention.

FIG. 5 illustrates a prototype dosage form fabricated in accordance with principles of the present invention.

FIG. 6 is a graph of drug concentrations versus saturations in accordance with principles of the present invention.

FIG. 7 is a graph of dosage per unit volume in accordance with principles of the present invention.

FIG. 8 illustrates a single microvalve for dispensing a suspension in accordance with principles of the present invention.

FIG. 9 illustrates a manifold for multiple microvalves in accordance with principles of the present invention.

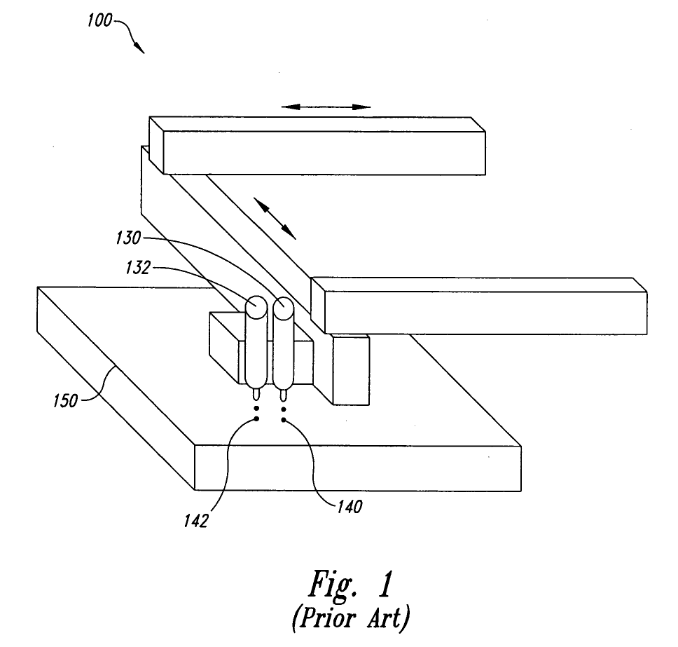

### DETAILED DESCRIPTION OF THE INVENTION

The invention includes dispensing a suspension containing solid particles for use in manufacturing a dosage form or other biomedical article by 3DP. A suspension contains solid particles suspended in a liquid. The solid particles may be particles of material that are insoluble in the liquid, or they may be particles of a substance that have already dissolved in the liquid up to the saturation level and are present in a concentration beyond what can be dissolved. A substantially insoluble substance can be considered to be a solubility of less than one part in 10,000. In addition to solid particles, the liquid may also contain other substances dissolved in it, either substances containing Active Pharmaceutical Ingredients (API) or substances without API.

In the 3DP process, binder liquid is dispensed onto the bulk powder material. One possible purpose of the binder is to carry the desired substances, which may be particles of a solid substance such as API, to the powder, in selected places and in selected quantities. Another possible purpose is to cause particles to bind to each other. The binder liquid may further serve both of these functions or some portion thereof. Binding of the particles can occur through several mechanisms, for example, when the binder liquid acts as a solvent of the bulk material or powder, in which case the liquid actually dissolves powder particles. As the solvent in the liquid evaporates, the particles resolidify such that they are joined together. Another mechanism is that the binder liquid simply solidifies around solid particles or solidifies such that it is connected to solid particles, thereby binding them. The binder liquid may contain a dissolved binding substance that is left behind when the volatile part of the binder liquid evaporates, which solidifies around solid particles or solidifies such that it is connected to solid particles, thereby binding solid particles together. The dissolved substance may be an inorganic substance or a low molecular weight (non-polymeric) organic substance.

In accordance with aspects of the current invention, the binder fluid is a suspension containing solid particles. As a result of the presence of solid particles, steps may be taken to guarantee that the solid particles remain uniformly distributed and suspended in the liquid. A principal variable determining how well particles stay in suspension is the size of the particles. The smaller the particles, the better able they are to remain suspended by virtue of Brownian motion. In order to encourage stability of suspensions, the particles may be in the range of less than or equal to 5 microns average dimension and greater than or equal to 100 nanometers average dimension. In order to achieve a narrow particle size distribution, dry milling or, more commonly, wet milling may be used. A higher viscosity fluid will also assist in keeping the solid particles uniformly distributed and suspended in the liquid.

The benefits of small particle size also imply the desirability of preventing particles from agglomerating, because agglomeration would effectively increase their size and cause or accelerate settling of the combined particles. Prevention of agglomeration can be accomplished with one or more of several categories of additives to the suspending liquid. One type of suspending agent is a steric hindrant. A steric hindrant is a molecule that attaches to the surfaces of particles through chemical absorption. The molecule has chains or groups that take up space around the particle, and prevent close approach of another similarly “coated” particle. Since the particles are prevented from touching, no agglomeration can occur, and the suspension remains stable. An example of such an additive is polyvinyl pyrrolidone (PVP).

In addition to preventing the particles from agglomerating or sticking together, in API suspensions, surfactants and dispersants are used to manipulate the surface charge. In a surfactant or dispersant, the molecules act to maintain a suspension by manipulating the surface charge of the particles and creating electrostatic repulsion between the particles. This electrostatic repulsion prevents agglomeration of the slurry or suspension. The surface charge of the particles in API suspensions are particularly difficult to manipulate because the organic molecules that make up an API particle can often possess positive and negative surface charges under different conditions, and may even have positive and negative areas of the same particle. This is contrasted with, for example, a ceramic particle that has a uniform surface charge. A suspending agent such as Avicel RC-591(10% Na CMC (sodium carboxylmethylcellulose), 90% microcrystalline cellulose) may be used with API suspensions.

Even with the benefits of such additives, there are limits as to how high a solids content can be created and maintained in a suspension. There are two possible limitations or criteria. One is due to the fact that the apparent viscosity of a fluid changes, by typically increasing, as the content of suspended solids changes. The viscosity of the suspension even with particles present should remain within a range suitable for dispensing by a particular dispensing technology or printhead, such as typically 0.3 to 20 cP. The suspension for use in the present invention may be formulated to remain within such a range. However, this is not typically a governing limitation or criterion.

The other limitation or criterion is a solids content at which agglomeration can begin to occur even with the use of steric hindrants, surfactants, suspending agents, and the like. For many suspensions, this limit is around 40%-50% by volume solids content, with some dependence on the material being dispersed, dispersing agents, suspending medium, and the like. The suspension for use in the present invention is formulated to remain below this limit.

The suspension may further contain solubilized Active Pharmaceutical Ingredient. By solubilization, compounds that are typically insoluble can form micelles to increase the solubility in the dispersing system when surfactant or solubilizer is added to the system. Surfactants form aggregates of molecules or ions called micelles when the concentration of the surfactant solute in the bulk of the solution exceeds a limiting value, the so-called critical micelle concentration. The formation of micelles is referred to herein as a solubilization process.

The ability of a suspension to carry solids content up to a viscosity or dispersion limit permits delivery of a much larger concentration of desired substance such as API via the binder liquid than is possible with solution printing, where the concentration is limited to somewhat less than the saturation concentration of solubility.

Higher concentration of API delivered to the dosage form is one aspect of the present invention. Another aspect is increased bioavailability of the API in the dosage form. All API have a bioavailability that describes how much of the compound enters the recipient's bloodstream for a given administered dose. One method of increasing the bioavailability of the API is to alter the structure of the API. An amorphous API has a greater aqueous solubility than the corresponding crystalline material of identical chemical composition, and so has a greater bioavailability. Greater bioavailability can mean reduced use of expensive pharmaceutical materials, and control of bioavailability provides improved control over the dose actually received by the patient's bodily tissues. The difference in bioavailability for amorphous API compared to crystalline versions of the same API has been shown to be as much as a factor of 5, with the amorphous material having greater bioavailability.

Wet dispensing of the API as a microfine suspension or in solubilized form allows a solid dosage form to include an API in an amorphous state. Providing a drug in an amorphous state is advantageous because it results in a drug with higher bioavailability to the patient than a drug that is allowed to exist in a crystalline form. The body better absorbs drugs in an amorphous, non-crystalline state than drugs in a crystalline state due to the higher surface area for dissolution and absorption. Further, as contrasted to solution printing, in suspension printing, when an API powder is prepared to include API in an amorphous state, the API will remain in the amorphous state in the dispensed product. In solution printing, it is unclear whether the API will be in the amorphous or crystalline state because the API must go through a dissolution phase followed by a resolidification phase. The variability of the API state in solution printing results in significant variability in the bioavailability of the active and has thus limited the use of an amorphous state for API that are solution printed.

Another aspect of the present invention includes an API that is soluble in water but insoluble in ethanol or other alcohols such that an API in an amorphous state, or a crystalline state if desired, could be dispensed in an ethanol suspension, leaving the crystallinity or amorphousness unchanged. A substantially insoluble substance can be considered to be a solubility of less than one part in 10,000. Examples of such APIs include but are not limited to: hydromorphone hydrochloride, pilocarpine hydrochloride, and tranylcypromine sulfate.

Yet another aspect of the present invention includes printing multiple passes of the suspension described above in order to further increase the API loading of the dosage form in a select region or over the entire dosage form.

The invention is further described, but is in no way limited, by the following examples.

### EXAMPLE 1

### Dosage Form Printed by a Continuous-Jet Printhead

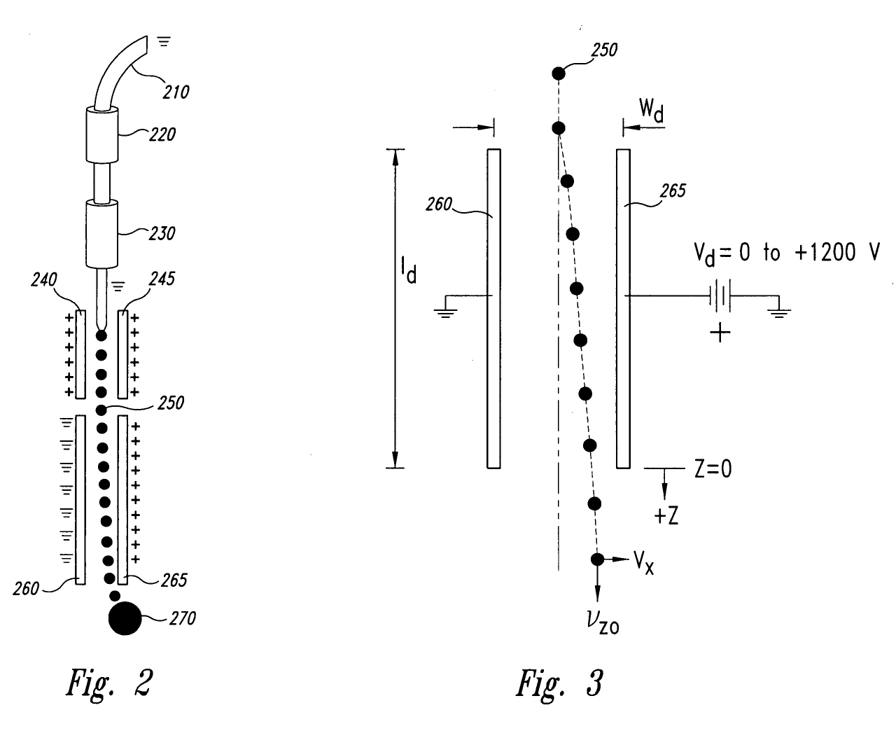

FIG. 2 illustrates a continuous jet with deflection printhead dispensing a suspension that may contain a significantly large solids loading. The continuous jet printing used in fabricating this embodiment of the pharmaceutical form is called CJ Charge and Deflection Printing, or CJ/CD. A continuous stream of pressure-driven flow may be modulated using an excitation device located close to the orifice, resulting in a controlled droplet break off. Individual droplets are either allowed to travel to the powder bed, or are instead “caught” by an electronic printhead that applies a charge to droplets and then deflects them selectively into a vacuum collection system where they may be recycled.

The first of these steps was stream modulation. The fluid 210 was forced through a piezoelectric tube actuator 220 that was connected to a function generator (not shown). The piezoelectric actuator of the present invention operates at 30-60 KHz. The mechanical vibration introduced into the fluid stream was used to control droplet break off upon exiting the orifice 230. The orifice opening in this embodiment was approximately 50 μm (microns).

In order for droplets to be controlled using computer design, the droplets are charged electrostatically. The jet was continuous up until break off, and was thus in contact with the grounded printhead and machine. Below the point at which droplets break up, they were isolated from one another. The stream was passed between two parallel charging plates 240, 245 such that break off occurred between the plates 240, 245.

The two charging plates 240, 245 could be charged or uncharged. The charge in this embodiment was +110 volts. The charging cell is “on” when the plates are charged positively. Droplets take on a negative charge upon break off between the plates when the charging cell is “on”. The stream is grounded, and the droplets become negatively charged upon break off as the positive field in the cell attracts the negative ions down stream. The charging cell is “off” when the plates are neutral or uncharged. Droplets remain neutral in this state.

The charging plates 240, 245 were designed to accommodate the longer break off lengths that correspond to organic solvents, as well as the traditional aqueous based binder fluids for the purposes of printing pharmaceutically relevant solutions and suspensions.

FIG. 3 illustrates an enlarged view of the deflection plates and the deflected drops shown in FIG. 2. Droplets 250 exiting the charging plates then traveled between two parallel deflection plates 260, 265. One deflection plate carried a variable net positive charge of up to 1200 volts. The opposite plate was grounded and was therefore neutral. Droplets exiting the charging cells that have not been charged, for example, when the charging cell is “off,” passed through this asymmetric charge field and continued straight to the powder bed to be printed. Thus, when the charging cell was “off,” the printhead was dispensing fluid to the powder bed below. Droplets exiting the charging cells with a negative charge, for example, when the charging cell is “on,” were deflected towards the positive deflection plate. A cylindrical vacuum catcher 270 was located below the positive plate and directly in the path of a deflected stream. A deflected stream of droplets wetted this cylindrical vacuum catcher and was vacuumed into a collection unit for later recycling. In the operation of a CJ/CD printhead, typically much of the liquid is recycled rather than being printed onto a print job.

The printhead was designed for individual operation of four fluid jets, and allowed for individual fluid recycling which is important when simultaneously printing and recycling various binder solutions, excipients, and drugs. It was made of Teflon and stainless steel.

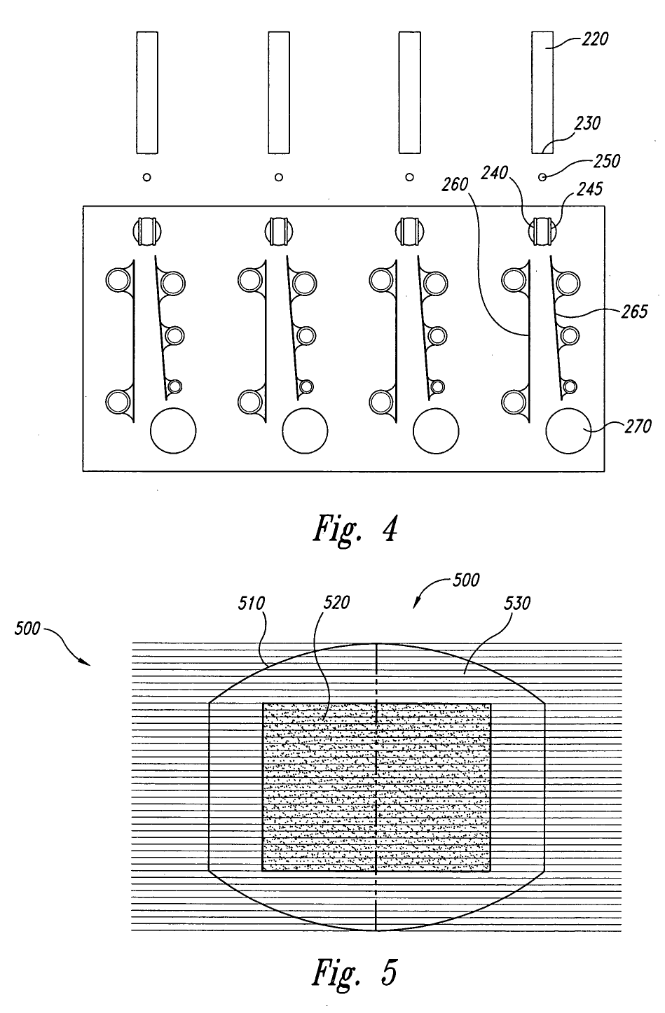

FIG. 4 illustrates one embodiment of a Continuous Jet Charge Deflection Printhead (CJ/CD) with multiple printheads of FIG. 2.

The powder used in fabricating these samples was 50-wt % microcrystalline cellulose (Avicel PH301) (particle size between 38 and 53 microns) mixed together with 50-wt % lactose (53-74 microns), with a packing fraction of 0.428, and using a layer height of 200 microns. The drops were printed through a nozzle of 50 micron orifice diameter, and droplets were optionally charged and deflected to control whether individual drops were printed into onto the powder bed.

The results presented herein represent the first time this technique has been introduced to printing pharmaceutical materials. The suspension was an aqueous suspension containing either 22 wt % or 41.5 wt % naproxen (Nanosystems, Inc.) suspended in water. Naproxen is (S)-6-Methoxy-alpha-methyl-2-naphthaleneacetic acid, or C₁₄H₁₃NaO₃. Naproxen is soluble in water, but the suspension used here contained fine powder particles of the drug each coated by an insoluble coating, so the effect was like having particles which were themselves insoluble particles. Suspending agents were also present.

No particular problem was observed as far as buildup of any substance at the catcher. Runs were performed for several hours at a time. As is typical for CJ/CD printheads, the vast majority of the liquid was not printed but rather was deflected and caught at the catcher.

FIG. 5 illustrates a prototype dosage form 500. The prototype dosage form fabricated in the current embodiment comprised an outer non-API-containing region 530 that surrounds an inner API-containing region 520. The use of the non-API-containing outer region 530 was intended for other purposes and was not actually necessary for demonstrating suspension printing or quantifying its results. When concentration of delivered API is reported herein, it is the concentration of the API contained in the API-containing region 520, not a concentration averaged over the entire dosage form 500. The printed article 500 illustrated in this embodiment of the present invention includes rounded caps on a central cylindrical region 520.

As shown in FIG. 5, the dosage form 500 of the present embodiment is constructed in a symmetrical geometry with 9 layers making up the top curved surface, 9 layers making up the bottom curved surface, and 25 center layers making up the API containing region, for a total of 43 layers. The layers are 200 microns layer height, with a line-to-line spacing of 120 microns, a drug-printed region 7 mm in diameter, and saturated to a saturation parameter of 1.0. The outer region of the dosage form was printed with a solution of 5-wt % Eudragit L100 (Rohm Pharma) in ethanol. The Eudragit L100 served as a binder substance that, upon evaporation of the volatile solvent, binds particles together by solidifying around adjacent particles or by solidifying so as to form necks at and near the contact points of adjacent particles.

The interior API region was printed with a binder liquid containing API and a marker substance. In this region the binder liquid did not actually contain a binder substance because the binder substance used to print the surrounding outer region held the outside of the dosage form together. The API was 22-wt % naproxen (Nanosystems, Inc.) suspended in water. In another embodiment, such a suspension was printed with a solids content of 41.5 wt % Naproxen. Naproxen is actually soluble in water, but the suspension used here contained fine powder particles of the drug each coated by an insoluble coating, so the effect was of insoluble particles. The suspensions contained naproxen particles approximately 200-500 nanometers in size, coated with a substance to render them insoluble. The suspension further contained approximately 0.1 w/w % PVP for steric dispersion in deionized water. The suspensions were first filtered, and then measured for wt % solids loading. A saturation of 1.0 was used to print the API region.

After completion of printing, the tablets were dried for two days in a nitrogen glove box, and then the excess powder was removed with an air de-duster. For measurement of API content, the dosage forms were allowed to completely dissolve in 900 mL of phosphate buffer solution with pH 7.4 at 37° C. Absorbance was then measured using a spectrophotometer.

The first group of dosage forms, printed with the 22-wt % naproxen suspension, was measured to contain 26.7 +/−0.7 mg as determined spectrophotometrically. The density of API in the API-containing region of the as-printed tablets was δ=139.1 mg/cc.

The second group of dosage forms, printed with 41.5-wt % naproxen suspension, was measured to contain 50.0 +/−0.8 mg as determined spectrophotometrically. The density of API in the API-containing region of the as-printed tablets was δ=293.2 mg/cc.

Table 1 summarizes the results from the fabrication of dosage forms using the naproxen suspension.

TABLE 1
δ Values for the drug-containing regions of dosage forms (mg/cc)

| Concentration of | 22 wt % | 41.5 wt % |
|---|---|---|
| suspension | naproxen | naproxen |
| Density of | 139.1 | 293.2 |
| deposited API | | |

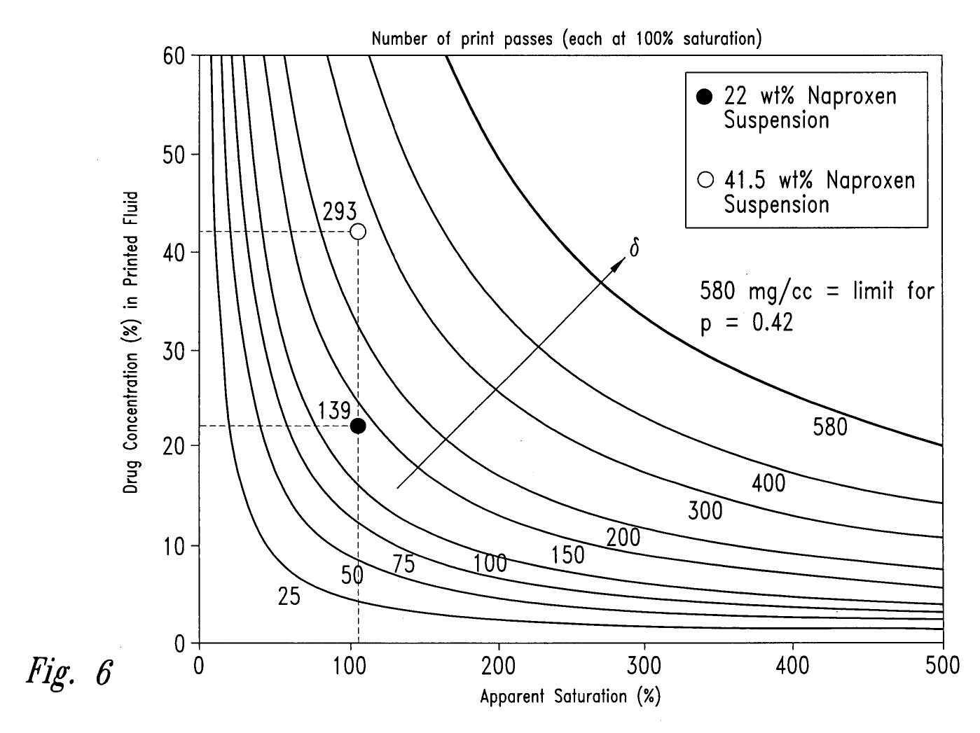

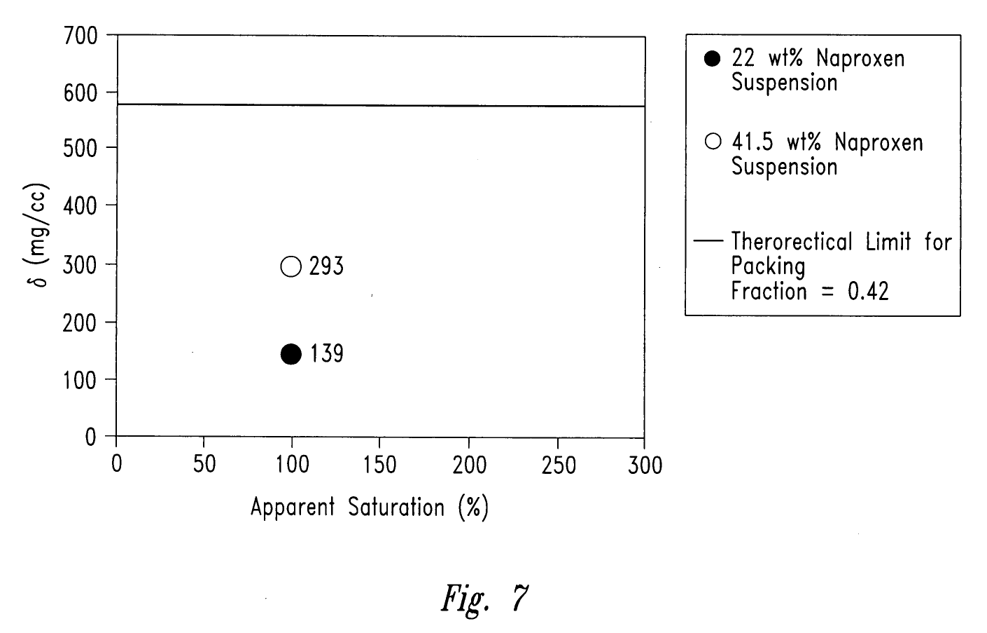

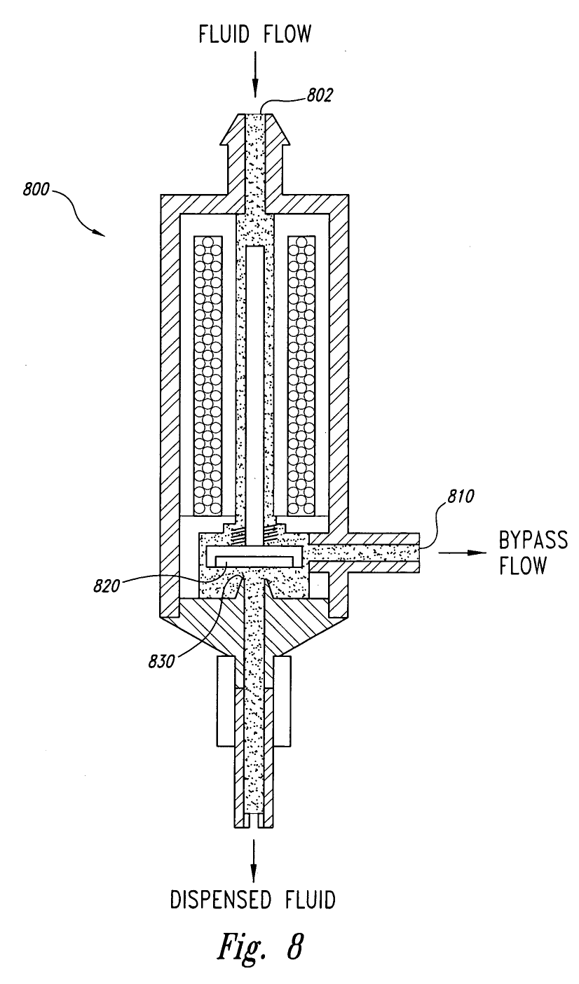

FIG. 6 shows the results for experimentally measured dosage per unit volume, δ, for the various as-printed dosage forms shown on a plot with calculated δ contours. FIG. 6 shows the results for detected dosage per unit volume, δ, for each of the above as-printed tablets, as compared to the calculated δ contours for a powder with packing fraction of 0.42.

Thus, in the case of printing with the more concentrated suspension, this represents attaining a drug concentration which is approximately 50% of the theoretical limit, or filling approximately 50% of the total originally available void volume.

FIG. 7 also shows the δ values achieved in this study. The largest value achieved was achieved for the 41.5-wt % naproxen suspension printed at 100% saturation, and was a δ of 293.2 mg/cc

In general, it is possible to further increase the loading of deposited solids such as API by printing binder liquid or suspension a first time on a deposited layer of powder, allowing the printed layer to dry at least partially, and re-printing the same places on the layer in another pass, and if necessary repeating the process still further. It would also be possible, in multi-pass printing, to double-print in some places while single-printing in other places, thereby achieving a gradient, or in general, to print unequal numbers of re-printings at different places to differentially load the dosage form. Variable drop volume printing, if available from a particular dispensing technology, could also be used for this purpose.

High δ values (mg_{drug}/cc_{drugregion}) are desired for printing high dosage forms. Tablets with high δ concentrations can be printed smaller while maintaining the same tablet dosage as those with low δ concentrations. The use of high solids loading suspensions increased the dosage per unit tablet volume considerably.

### EXAMPLE 2

### Dosage Forms Suspension-Printed with a Microvalve

In this example, the dispensed binder liquid was a somewhat dilute suspension containing an insoluble API, and it was dispensed through microvalves, namely, miniature solenoid valves. The microvalves dispensed through nozzles that were holes drilled through jewels.

The valve operates with a plunger forming a seal against an elastomeric seat, and therefore, a good seal is needed to ensure precision dispensing. The particles in the suspension of the present embodiment did not interfere with the seal of the plunger against the elastomeric seat. Further, the dilute suspensions of very fine particles of the present invention did not appear to damage the seat of the valve or other parts that are involved in forming the seal.

The API used was camptothecin (C₂₀H₁₅N₃O₆) and its derivative, 9-nitrocamptothecin (9-NC) (rubitecan). These drugs are substantially insoluble in water. Microfine camptothecin or 9-NC was incorporated into the suspension at a concentration of 2.5% (by weight). The average particle size was approximately 0.5 microns. Other substances included in the suspension were Avicel RC-591 (10% Na CMC (sodium carboxymethylcellulose), 90% microcrystalline cellulose) and PVP K-25 (polyvinyl pyrrolidone of a molecular weight of 25,000 g/mole), which function as a suspending agent and steric hindrant to prevent agglomerate formation, respectively. It is estimated that suspensions with a solids concentration of up to approximately 5-wt % could be dispensed through microvalves.

The powder that was used to make the ODF matrix (the powder upon which printing was performed) was a mixture containing hydroxypropylmethyl cellulose (HPMC) and other excipients, such as Avicel CL-611, Avicel PH-301 and lactose. Avicel is manufactured by the FMC Corp., Philadelphia, Pa. Avicel CL-611 contains 85% of microcrystalline cellulose and 15% of sodium carboxymethyl cellulose (Na CMC). Na CMC functions as a solid binder that gels upon hydration. Avicel PH-301 is a type of microcrystalline cellulose, a water-insoluble excipient. HPMC is a gelation agent. The quantity of HPMC can be varied to adjust the drug release rate. Addition of more HPMC effectively decreases the drug release rate. Flow rates of drug suspensions were adjusted to deliver a nominal total drug loading of 0.5 mg active to the core region of the ODF.

For the present application, the active agent or drug was deposited in a central region or core of the dosage form. The liquid for this deposition is herein referred to as the core binder. The core binder may also function as a binding substance, thus causing powder particles to adhere together, but it is not essential that it function as a binding substance. The liquid may simply serve as a means of placing the drug within the dosage form.

In addition to the already described suspension, the printhead also dispensed another liquid, which was used to surround an API-containing core region with an enclosure or surrounding layer or wall. This geometry may be useful for time release or other purposes. This other binder liquid did not contain API and was not a suspension.

The suspension may be dispensed onto the powder in such as way as to create a nonuniform distribution of concentration of API. In some instances, it may be desirable to create a gradient of concentration. In other instances, it may be desirable to create some portion of the ODF containing essentially none of the API in the suspension. For example, the region containing essentially no API may be in the form of an enclosing region that on all sides surrounds the API-containing region, or interior walls may be created within the API containing core region. The enclosing region may serve purposes such as controlling time release or isolating the interior from the outside world. All of this is possible by appropriate programming of the dispensing of one or more liquids during the 3DP process. For gradients, the suspension can be dispensed with variable drop volume, if the printhead allows, or it can be dispensed with varying numbers of reprints of an individual layer. A second binder liquid may be dispensed from a second dispenser if available. A microvalve can deliver variable a drop volume by appropriate adjustment of the pulsewidth of the driving electrical signal supplied to the microvalve.

In yet another embodiment, the binder may contain an active in solubilized form. In general, a binder liquid may optionally contain both suspended solid particles of one API and another API substance dissolved in the same liquid.

Wet dispensing of the toxic or potent drug in a solution, microfine suspension, or in solubilized form allows a solid dosage form to include a toxic or potent drug in an amorphous state. Providing a drug in an amorphous state is advantageous because it results in a drug with higher bioavailability to the patient than a drug that is allowed to exist in a crystalline form. The body better absorbs drugs in an amorphous, non-crystalline state than drugs in a crystalline state due to the higher surface area for dissolution and absorption. In suspension printing, when an API powder is prepared to include API in an amorphous state, the API will remain in the amorphous state in the dispensed product. In contrast, in solution printing, it is unclear whether the API will be in the amorphous or crystalline state because the API must go through a dissolution phase followed by a resolidification phase. The variability of the API state in solution printing results in significant variability in the bioavailability of the active and has thus limited the use of an amorphous state API in solution printing. When the drug is in amorphous form with the presence of crystallization inhibitors, crystal growth can be inhibited, thus enhancing the absorption of the drug. Steric hindrants, such as PVPs, HPMCs, or surfactants in a binder solution that contains the active will inhibit the recrystallization of the active in the dosage form after drying. Therefore, the resolidifed active particles will either be in amorphous form or have very small crystal size. As a result, the absorption will be enhanced as compared to the original solid state of the active because the increase in surface area for the dissolution and hence absorption will enhance the bioavailability of the drug.

Currently, the use of API in the amorphous state is relatively limited because there has not previously been a good method of achieving amorphous state API in a dosage form. Aspects of the current invention provide a method of achieving amorphous state API in a dosage form. Many solid materials exist as crystals, which have long-range order in the arrangement of molecules or atoms. The amorphous state is another state in which solid materials can exist, and it is a state that exhibits no long-range order in the arrangement of the molecules. Normally, for solid materials, the crystalline state is the lowest energy state possible and hence is energetically preferred. The amorphous state is of a higher energy and so is metastable.

Amorphous materials will revert to the crystalline state under certain conditions, which include elevated temperature and certain humidity conditions. However, under certain conditions, the amorphous state can persist for extremely long periods of time. Probably the most common example of an amorphous material is glass. Various solid materials that are normally thought of as crystalline can also exist in an amorphous state, including metals and pharmaceutical compounds. Attainment of the amorphous state is frequently associated with some sort of rapid formation mechanism that does not allow enough time for crystals to form. Alternatively, grinding crystals to extremely small particle sizes can produce behavior characteristics of the amorphous state.

### EXAMPLE 3

### Continuous Suspension Circulation Through Manifold

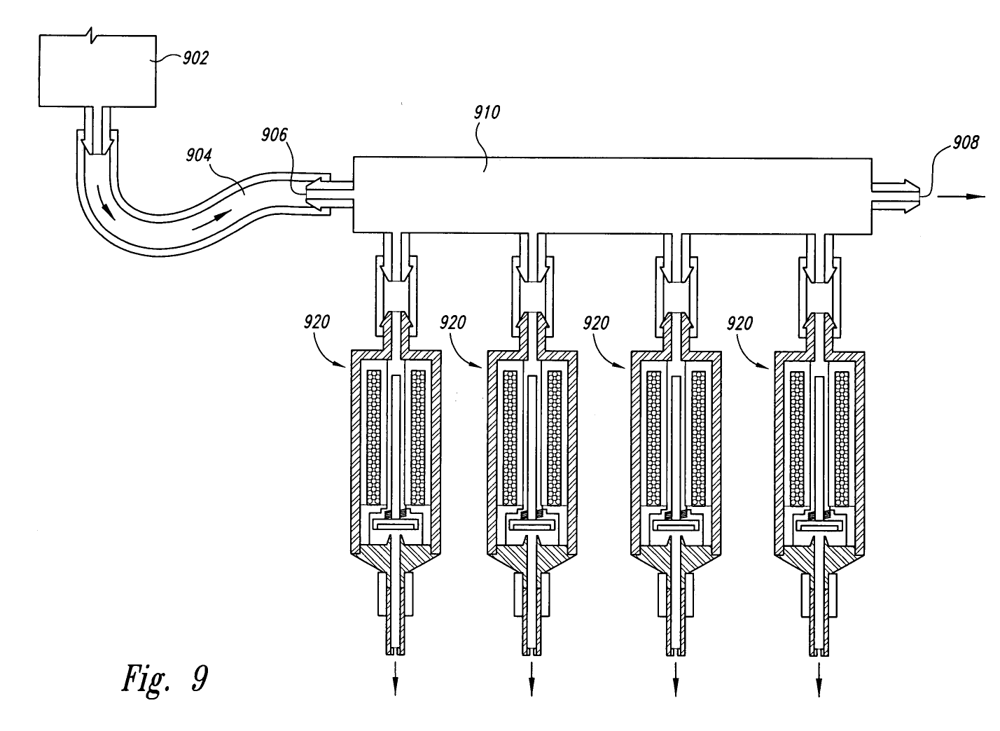

For printing suspensions through a microvalve, or in general through any type of dispenser, it is believed to be helpful to provide a flow geometry such that the suspension can stay in motion, by means of flow-through or bypass flow geometry, to a point as close as possible to the location of the actual valving action. This discourages settling-out of the suspended particles. FIG. 9 illustrates a flow-through manifold which supplies multiple microvalves (similar to the microvalve of FIG. 8) connected in parallel.

As illustrated in FIG. 9, a plurality of valves 920 draw their fluid from a manifold 910, and the fluid in the manifold 910 is in continuous motion as a result of an open flowpath through the manifold 910. A suspension is supplied from a fluid source 902, which may be maintained at an elevated pressure through a supply line 904 to the inlet end 906 of a manifold 910. Connecting to the manifold 910 are a plurality of individual valves 920 which can receive fluid from manifold 910 and dispense it to the target or desired application. Within its body, manifold 910 may generally define a flow path from inlet end 906 to an outlet end 908 located substantially away from and opposite the inlet end 906. Outlet end 908 may be always open so as to establish a substantially continuous flow of fluid through the manifold regardless of whether any or all of the valves 920 are receiving fluid from the manifold 910. The fluid which leaves through the always-open outlet from the manifold may be returned to source 902 for later re-use, either by action of a pump or on an occasional basis when source 902 is depressurized. The flowrate of fluid through the always-open flowpath may be such as to prevent settling out of suspended particles inside the manifold 910.

### EXAMPLE 4

### Continuous Suspension Circulation Through Valve

This Example uses the same principle as Example 3, but keeps the fluid circulating to a point that is even further downstream, namely, very close to the valve seat. In this embodiment, the valve 800 may have a bypass exit 810 located within the body of the valve itself as is illustrated in FIG. 8. Flow is dispensed by the action of valve 800, shown as being solenoid-operated. The motion of moving part 820 relative to valve seat 830 produces this valving action. The liquid being dispensed enters the valve 800 at an entrance port 802, which is located some distance away from the place where moving part 820 seats against seat 830. The use of bypass flowpath 810 provides continuous fluid motion very close to the point where flow is actually turned on or shut off for dispensing through the dispensing flowpath. A microvalve of conventional design may be modified by drilling a hole from the exterior and inserting and securing a tube appropriately, such that the bypass flowpath is established, or such a flowpath could be designed into a valve body from the beginning.

In an alternative embodiment, the embodiments of Example 4 and of Example 3 are combined, for example, to have a bypass from the manifold and also a bypass from individual valves. The necessity of either or both of these strategies depends on the fluid properties of a particular suspension, particle size, settling or sedimentation rate of the particles, and the like.

Furthermore, alternative valves may be used instead of the microvalves illustrated in FIGS. 8 and 9. For example, a piezoelectric drop-on-demand dispenser (PZDOD) may be used in accordance with aspects of the present invention. Piezoelectric drop-on-demand dispensers are known in the art. The PZDOD does not include the moving part 820 shown in FIG. 8. With respect to maintaining the solid particles in suspension, similar apparatus may be used to continuously flow the suspension through the valve as described above.

### EXAMPLE 5

### Biomedical Articles

So far the examples have described manufacturing of Dosage Forms using suspensions containing API. Dosage forms include Oral Dosage Forms, implantables and others. An ODF is not the only type of article that may be usefully manufactured according to the present invention, and API is not the only type of insoluble or lightly soluble additive that may be of interest to dispense or print. It is also possible to manufacture other biomedical articles. Such biomedical articles include but are not limited to implantable devices such as implantable drug delivery devices, surgical leave-behinds, and other implants; bone substitutes; and tissue scaffolds which serve to host the ingrowth of cells and tissues.

In such cases, there are other categories of solid substances, besides Active Pharmaceutical Ingredients, which may be desired to be included in the form of the solid particles contained in a suspension. In some of these applications, it may be desirable to incorporate, into the 3DP printed article, any of a variety of substances such as substances that promote the growth of bone or other tissues. Such substances can include cells, cell fragments, cellular material, proteins, growth factors, Active Pharmaceutical Ingredients, at least some of which are insoluble in typical solvents, bone particles, cartilage particles, or other biological or inert materials that are insoluble or nearly insoluble. For example, it may be desirable to include in the dispensed suspension fine particles of the same material as the powder in the layer of powder, perhaps much finer than the particles that are actually spread to make the powder bed. A material that may be used in such a way is nanocrystalline hydroxyapatite, which may be used with larger particles of hydroxyapatite in the manufacture of bone substitutes in order to create higher density parts.

Inclusion of such fine particles can help to fill in the empty spaces between particles of the spread powder. If a sintering step is involved, the extremely fine particles may help to create better necks bridging gaps between the spread powder particles, thereby increasing the strength of the eventual sintered part. The use of very fine particles together with larger particles can also help to improve surface smoothness of a 3DP printed part, and this can be accomplished by dispensing the fine particles as part of a suspension. The dispensed liquid may include a binder substance in addition to the suspended solid particles.

In any such application, it may be desirable to create nonuniform concentration; wherein the concentration of the solid particle substances suspended in the suspension varies from one place in the 3DP printed biomedical article to another place. Such a nonuniformity can take the form of a concentration gradient. It can also take the form of having some regions having an essentially zero concentration of the suspended substance(s) and other regions having desired concentrations of the suspended substance(s).

It should be understood, in any reference herein to local composition, that the local composition is to be measured or calculated on the basis of being averaged over a size scale which is somewhat greater than the size of individual powder particles or particles of suspended solid.

### EXAMPLE 6

### Non-Medical Applications

The described use of a microvalve with a suspension, and the described design of the microvalve with bypass, can be extended to essentially any material that can be created in the form of a suspension. This has applicability to three-dimensional printing for non-medical purposes as well, wherein the solids suspended may be particles of ceramic, metal, pigment, or other substances. Such suspension printing may be done with the aid of a bypass flowpath within the valve itself as described in Example 4, or with the aid of bypass by means of a manifold as described in Example 3, or with no form of bypass.

Further Discussion

The printing of suspensions is not limited by a solubility limit, and therefore can be printed with concentrations up to the viscosity limit or up to the dispersion limit. Highly concentrated drug suspensions can be printed in accordance with aspects of the present invention. Examples of insoluble or lightly-soluble drugs which could be suspension-printed by the present invention, in addition to the examples already given, include ibuprofen, nitrofurantoin, acetaminophen, ondansetron, taxol, lovastatin, ciprofloxacin hydrochloride, sulfonamide (sulfamethoxazole), and others.

Microvalves can be used in a mode of printing variable drop volume using appropriate adjustment of the duration and/or shape of the electrical waveform driving the microvalves. With any of the dispensers described, it is possible to print multiple passes during a three-dimensional printing process, thereby achieving still higher loading of dispensed API or achieving spatial variation of the amount of API deposited. The described dispensers and printheads can be used for dispensing purposes other than 3DP, including dispensing chemical and biological substances for high throughput screening and combinatorial chemistry applications.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above.

The various embodiments described above can be combined to provide further embodiments. Aspects of the invention can be modified, if necessary, to employ the process, apparatuses and concepts of the various patents, applications and publications described above to provide yet further embodiments of the invention. All patents, patent applications and publications cited herein are incorporated by reference in their entirety.

These and other changes can be made to the invention in light of the above detailed description. In general, in the following claims, the terms used should not be construed to limit the invention to the specific embodiments disclosed in the specification and the claims, but should be construed to include all devices that operate under the claims to provide a method for dispensing a liquid. Accordingly, the invention is not limited by the disclosure, but instead the scope of the invention is to be determined entirely by the following claims.

We claim:

1. A method of manufacturing a biomedical article, comprising:
depositing a layer of powder;
dispensing, onto portions of the layer of powder, a suspension comprising solid particles of at least one substance selected from the group consisting of: cells, cell fragments, cellular material, proteins, growth factors, bone particles, cartilage particles, other biological or inert materials which are insoluble or nearly insoluble, Active Pharmaceutical Ingredients, and very fine particles of the same material as the powder in the layer of powder;
and repeating the above steps as many times as needed to produce the biomedical article.

2. The method of claim 1 wherein the suspension further comprises suspending agents and/or steric hindrants.

3. The method of claim 1 wherein the suspension further comprises one or more additional API dissolved in the liquid.

4. The method of claim 1 wherein the suspension further comprises one or more binding substances dissolved in the liquid.

5. The method of claim 1 wherein the biomedical article is an implantable device.

6. The method of claim 1 wherein the biomedical article is a bone substitute.

7. The method of claim 1, further comprising, after dispensing, allowing or causing the dispensed suspension to at least partially dry, and dispensing a second suspension containing solid particles of at least one Active Pharmaceutical Ingredient again onto portions of the layer of excipient powder, at least one additional time before depositing the next layer of excipient powder.

8. The method of claim 7 wherein the pattern of deposition during the second printing is different from the pattern during the first printing.

9. The method of claim 1, wherein at least some of the suspended solid particles comprise an amorphous form of Active Pharmaceutical Ingredient.

10. The biomedical article of claim 7 wherein the second substance has a local concentration at local places in the dosage form and wherein the local concentration of the second substance is nonuniform.

11. The biomedical article manufactured by the method of claim 1.

12. A biomedical article comprising:
a powder which is substantially insoluble;
a second substance selected from the group consisting of: cells, cell fragments, cellular material, proteins, growth factors, bone particles, cartilage particles, other biological or inert materials which are insoluble or nearly insoluble, Active Pharmaceutical Ingredients, and very fine particles of the same material as the powder in the layer of powder.

13. A method of three-dimensional printing, comprising dispensing a suspension through a solenoid-operated valve onto powder.

14. The method of claim 12 wherein the valve includes a valve body and within the valve body a seat and a moving part, and a bypass flowpath.

15. The method of claim 12 further comprising flowing suspension continuously through a manifold, and wherein the microvalve is supplied from the manifold.

16. The method of claim 13, wherein the solenoid-operated valve has a valve body and within the valve body a seat and a moving part adapted to fit against the seat and thereby close a flowpath, and further comprising a bypass path emanating from the valve body close to the valve seat, the bypass path being always open.
